(19) United States
(12) Patent Application Publication
(10) Pub. No.: US 2024/0206862 A1
(43) Pub. Date: Jun. 27, 2024

(54) INTELLIGENT ARTICULATION MANAGEMENT FOR ENDOLUMINAL DEVICES

(71) Applicant: Covidien LP, Mansfield, MA (US)

(72) Inventors: Nathan J. Knutson, Long Lake, MN (US); Evan M. Gustafson, Sioux Falls, SD (US); John W. Komp, Dillon, CO (US); Jesse J. Pischlar, St. Paul, MN (US)

(21) Appl. No.: 18/288,547

(22) PCT Filed: May 19, 2022
(86) PCT No.: PCT/IB2022/054649

§ 371 Date: Oct. 26, 2023

Related U.S. Application Data
(60) Provisional application No. 63/193,409, filed on May 26, 2021.

(51) Int. Cl. A61B 17/00; A61B 34/00; A61B 34/10; A61B 34/20; A61B 34/30; A61B 90/00; A61M 25/00; A61M 25/01

(52) U.S. Cl. CPC A61B 17/00234; A61B 34/20; A61B 34/25; A61B 34/71; A61B 2017/00323; A61B 2034/105; A61B 2034/2051; A61B 2034/2055; A61B 2034/301; A61B 2090/3937; A61M 2025/0008; A61M 25/0113; A61M 25/0127; A61M 25/0147; A61M 2025/0166

(57) ABSTRACT

A catheter system and method, the system including a catheter having a lumen, a position sensor, and at least one pull-wire to articulate the catheter, a tool including a metal tip at the distal end of the catheter, a proximity sensor associated with the catheter and configured to detect the presence of the tool proximate an articulation region on the target, and an articulation mechanism configured to apply pressure to the pull-wire to articulate and de-articulate the catheter. The method determining that the tool cannon pass through the articulation region based on the level of articulation of the catheter and the properties of the tool, de-articulating the catheter to allow the tool to pass through the articulation region and re-articulating the catheter to a position and orientation of the catheter prior to de-articulation.

### BACKGROUND

### Technical Field

This disclosure relates to the field of catheter navigation of and systems and methods for determining the location of a tool in a catheter, adjusting the articulation of a catheter to allow a tool to pass the curvature, and to prevent damage to the internal surfaces of catheter.

### Description of Related Art

There are several commonly applied medical methods, such as endoscopic procedures or minimally invasive procedures, for treating various maladies affecting organs including the liver, brain, heart, lungs, gall bladder, kidneys, and bones. Many of these procedures involve navigating a catheter to a point within the patient. Once arriving at the desired point, a tool such as a biopsy or therapy tool may be passed through the catheter to interact with the tissue at the point within the patient.

FIG. 8A depicts a catheter 102 having a position sensor 104 located at a distal end of the catheter 102. In FIG. 8B, as a tool 103 is advanced through the catheter 102, the rigidity of the tool 103 caused by for example the rigid metal tip of 105 of the tool 103 causes the metal tip 105 to impact the inner surface 107 of the catheter 102 as shown in greater detail in FIG. 8C. Such impact prevents the advancement of the tool 105 through the catheter 102. Further, the metal tip 105 can imbed and damage the inner surface 107, such damage stops the initial advancement of the tool 13 and can also cause issues with future advancement.

In addition, even where the tool 103 can be navigated through the curvature and not impact the inner surface 107 of the catheter 102, the friction caused by the curvature can at some point prevent further advancement. Similarly, because the metal tip 105 may be glued or press fit onto the catheter 109 of the tool 103 where the catheter 102 has an extreme curvature, the curvature can create sufficient friction on the metal tip 105 to remove it from the catheter 102 of the tool 103. In view of these challenges, particularly when navigating to points within the body which can only be reached following a tortuous path, improvements to current catheter navigation systems are desired.

### SUMMARY

One aspect of the disclosure is directed to a system for navigation of a catheter to a location within a patient including: a catheter including a lumen therethrough and a position sensor on a distal portion thereof; an articulation mechanism including at least one pull-wire for articulating an articulating region of the catheter; a tool for performing a procedure at a target site and translatable within the lumen of the catheter; a proximity sensor on the catheter to detect the tool is proximate the articulating region; and an application stored in a memory and executed by a processor of a computing device, the application when executed performing steps to determine properties of the tool based on a received an indication of a type of tool, record a position, articulation and vector to a target of the catheter, detect the tool in proximity to the articulation region, determine an amount of de-articulation necessary for the tool to pass through the articulation region, adjust the articulation of the catheter, detect the tool has passed the articulation region and is proximate a distal portion of the catheter, articulate the catheter to the recorded position, detect that the catheter has returned to the recorded position, and display an indication of realignment on a user interface. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The system where the application further performs a step of presenting an indication on the user interface of a required amount of de-articulation necessary to allow the tool to pass through the articulation region. The system where the articulation mechanism is a motorized mechanism. The system where the motorized mechanism is robotically controlled. The system where the motorized mechanism is manually operated. The system where the articulation mechanism is manually operated. The system where the application further performs a step of determining if de-articulation is required to remove the tool. The system where the application further performs steps of de-articulating the catheter and displaying an indication on the user interface that the tool can be safely removed. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A further aspect of the disclosure is directed to a method of placement of a tool proximate a target including receiving an image data set of a patient, generating a 3D model of a luminal network from the image data set. The method of placement also includes generating a pathway to a target location within the 3D model of the luminal network. The method of placement also includes detecting a catheter including a position sensor has been navigated to a position within the luminal network proximate the target location. The method of placement also includes receiving an indication of a type of tool being inserted into the catheter. The method of placement also includes determining specification data related to the type of tool being inserted into the catheter. The method of placement also includes recording the position, articulation, and a vector to the target location from a distal end of the catheter to the target location. The method of placement also includes detecting the position of the tool proximate an articulation region of the catheter. The method of placement also includes determining an amount of de-articulation necessary for the catheter to pass through the articulation region. The method of placement also includes adjusting the articulation of the catheter. The method of placement also includes detecting passage of the tool through the articulation region. The method of placement also includes articulating the catheter to the recorded position, articulation and vector to the target. The method of placement also includes displaying on a user interface an indication of realignment of the catheter with the target location. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method where determining specification data includes querying a look-up table in which the specification data is recorded. The method further including presenting an indication on the user interface of an amount of de-articulation necessary to allow the tool to pass through the catheter. The method further including manually manipulating a motor driven pull-wire system to de-articulate the catheter to achieve the presented amount of de-articulation. The method further including manually adjusting a pull-wire to achieve the presented amount of de-articulation. The method further including robotically driving a motor driven pull-wire system to de-articulate the catheter. The method further including determining if de-articulation is required to remove the tool. The method further including de-articulating the catheter and displaying an indication on the user interface that the tool can be safely removed. The method where the amount of de-articulation necessary is determined based on the determined specification data related to the type of tool inserted into the catheter. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Still a further aspect of the disclosure is directed to a catheter system including: a catheter having a lumen therethrough, a position sensor located at a distal end, and at least one pull-wire configured to articulate the catheter. The catheter system also includes a tool including a metal tip at the distal end. The catheter system also includes a proximity sensor associated with the catheter configured to detect a presence of the tool proximate an articulation region on the target. The catheter system also includes an articulation mechanism configured to apply pressure to the pull-wire to articulate and de-articulate the catheter; and a computing device, storing in a memory an application executable by a processor, the application when executed determining steps of determining that the tool cannot pass through the articulation region based on the articulation of the catheter and properties of the tool, de-articulating the catheter to allow the tool to pass through the articulation region, and re-articulating the catheter to a position and orientation of the catheter prior to de-articulation. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The catheter system where the proximity sensor is an optical sensor configured to read markings on the tool. The catheter system where the proximity sensor is an electromagnetic field sensor configured to detect interference in the electromagnetic field as the metal tip is advanced into proximity of the proximity sensor. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

Various aspects and embodiments of the disclosure are described hereinbelow with references to the drawings, wherein:

FIG. 1 is a schematic diagram of a system for navigating to soft-tissue targets via luminal networks in accordance with the disclosure;

FIG. 2 is a user interface of a navigation program in accordance with aspects of the disclosure;

FIG. 3A is a perspective view of a motorized catheter in accordance with the disclosure;

FIG. 3B is a detailed magnified view of a portion of the drive mechanism of the motorized catheter of FIG. 3A;

FIG. 3C is a perspective view of an articulating catheter in accordance with the disclosure;

FIG. 4 is a schematic view of a computing device in accordance with aspects of the disclosure;

FIG. 5 is a schematic view of a distal portion of a catheter having a sensor for detection of a tool;

FIG. 6 is a flow chart of a method for de-articulation and articulation to a desired location in accordance with the disclosure;

FIGS. 7A and 7B depict catheter being de-articulated to allow a tool to pass therethrough and re-articulation to an original position; and

FIGS. 8A and 8B depict a prior art articulated catheter 102 where the articulation prevents the tool from passing through the catheter.

### DETAILED DESCRIPTION

This disclosure is directed to systems and method of for determining the location of a tool in a catheter, adjusting the articulation of a catheter to allow a tool to pass the curvature, and to prevent damage to the internal surfaces of catheter.

In accordance with the disclosure, a 3D volume of a patient's lungs or another suitable portion of the anatomy, may be generated from previously acquired scans, such as CT scans. These scans may be used to generate a 3D model of the anatomy. The 3D model and related scan data are used to identify targets, e.g., potential lesions for biopsy or treatment, and to generate a pathway plan through the anatomy to reach the targets.

Once the pathway plan is generated and accepted by a clinician, that pathway plan may be utilized by a navigation system to drive a catheter along the pathway plan through the anatomy to reach the desired target. The driving of the catheter along the pathway plan may be manual or it may be robotic, or a combination of both. Manual systems include the ILLUMISITE navigation system sold by Medtronic PLC, robotic systems include the ION system sold by Intuitive Surgical Inc. and the MONARCH system sold by Auris Health, Inc. In a single procedure planning, registration of the pathway plan to the patient, and navigation are performed to enable a medical device, e.g., a catheter to be navigated along the planned path to reach a target, e.g., a lesion, so that a biopsy or treatment of the target can be completed.

As noted above, whether manual or robotic, the pathway plan and 3D model developed from the pre-procedure scan data may be registered to the patient before navigation of the catheter to a target within the anatomy can being. Once registered, a catheter or other tool may be navigated following the pathway plan to a desired location. Alternatively, the registration may occur as part of the navigation procedure where landmarks and other features (e.g., carina) and visual feedback using a camera are identified to collect sufficient data to generate a registration of the 3D model to the patient. While this registration is generally more that suitable for general navigation of the pathway, regardless of the registration method employed, and there are numerous registration methods, the 3D model and pathway plan may still not provide sufficient accuracy for target interaction allowing for the guidance of medical devices or instruments into the target for biopsy and treatment.

In some cases, the inaccuracy is caused by deformation of the patient's lungs during the procedure relative to the lungs at the time of the acquisition of the previously acquired CT data. This deformation (CT-to-Body divergence) may be caused by many different factors including, for example, changes in the body when transitioning from between a sedated state and a non-sedated state, the bronchoscope changing the patient's pose, the bronchoscope and catheter pushing the tissue, different lung volumes (e.g., the CT scans are acquired during full breath hold following inhale while navigation is typically performed while the patient is breathing), different beds, different days, etc. Thus, another imaging modality may be employed to assist in visualizing medical devices and targets in real-time and enhance the in-vivo navigation procedure.

In navigating the medical device to the target, clinicians may use a fluoroscopic imaging to visualize the position of the medical device relative to the target. While fluoroscopic images show highly dense objects, such as metal tools, bones, and large soft-tissue objects, e.g., the heart, the fluoroscopic images may not clearly show small soft-tissue objects of interest, such as lesions. Furthermore, the fluoroscopic images are two-dimensional (2D) projections which makes determining depths in the view difficult.

X-ray volumetric reconstruction has been developed to enable identification of soft tissue objects and to update the relative position of the target and the catheter in the pathway plan and 3D model. The volumetric reconstruction is made from a series of 2D fluoroscopic images taken at different angles to the tissue in question. In one method described in greater detail below, updating of the pathway plan and relative positions of the catheter and target can be achieved with a local registration process. This local registration process reduces CT-to-body divergence. After the local registration process, in one embodiment a locatable guide (i.e., a catheter with multiple sensors) may be removed from the catheter and a medical device, e.g., a biopsy tool, is introduced into the catheter for navigation to the target to perform the biopsy or treatment of the target, e.g., the lesion.

However, even with local registration where the relative position of the catheter and the target is updated in the 3D model and the pathway plan, maintaining the alignment of the catheter and the target as confirmed in the local registration can be challenging. The source of this challenge is related to two primary functions of the body, namely respiration and cardiac functions (i.e., heartbeat).

Another source of errors is the passage of tools through the catheter after local registration can cause tip deflection. When the catheter includes a sensor, e.g., an electromagnetic sensor or a flexible sensor (sensing shape and orientation of a portion of the catheter) these types of movements can be reported to the clinician via a graphic user interface (GUI) on which navigation software is displayed and allows for following the pathway plan to the target. Movements caused by the passage of tools through the catheter appear as movement of the target relative the position of the catheter on the GUI.

In accordance with embodiment of the disclosure the catheter 102 includes one or more pull wires. The movement of the sensor 104 is sensed by the navigation system and micro-adjustments can be made to the location of the catheter by manipulating the pull wires. These same pull wires can be employed to constantly adjust the position of the catheter, and specifically the distal portion of the catheter 102 as it moves within the lung. The pull wires cause the catheter 102 to change shape and curvature at the distal portion and may be manipulated either manually by a clinician or automatically via a computer-controlled system (e.g., a robot).

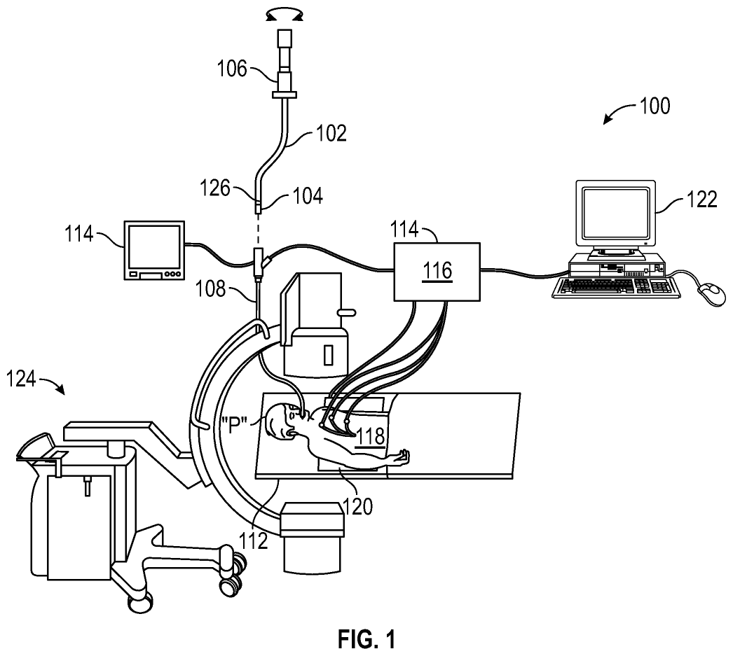

In accordance with aspects of the disclosure, and as noted above, the visualization of intra-body navigation of a medical device, e.g., a biopsy tool, towards a target, e.g., a lesion, may be a portion of a larger workflow of a navigation system, such as an electromagnetic navigation system. FIG. 1 is a perspective view of an exemplary system for facilitating navigation of a medical device, e.g., a catheter to a soft-tissue target via airways of the lungs. Those of skill in the art will recognize that other systems for navigation within a patient whether it be in the gastro-intestinal track (i.e., colon, intestines, stomach, and esophagus), vasculature, navigation within the thoracic and abdominal cavities (e.g., laparoscopic procedures) and others without departing from the scope of the disclosure.

System 100 may be configured to construct fluoroscopic based three-dimensional volumetric data of the target area from 2D fluoroscopic images to confirm navigation to a desired location. System 100 may be further configured to facilitate approach of a medical device to the target area by using Electromagnetic Navigation (EMN) and for determining the location of a medical device with respect to the target. One such EMN system is the ILLUMISITE system currently sold by Medtronic PLC, though other systems for intraluminal navigation are considered within the scope of the disclosure, as noted above.

One aspect of the system 100 is a software component for reviewing of computed tomography (CT) image scan data that has been acquired separately from system 100. The review of the CT image data allows a user to identify one or more targets, plan a pathway to an identified target (planning phase), navigate a catheter 102 to the target (navigation phase) using a user interface on computing device 122, and confirming placement of a sensor 104 relative to the target. The target may be tissue of interest identified by review of the CT image data during the planning phase. Following navigation, a medical device, such as a biopsy tool or other tool, may be inserted into catheter 102 to obtain a tissue sample from the tissue located at, or proximate to, the target.

As shown in FIG. 1, catheter 102 is part of a catheter guide assembly 106. In practice, catheter 102 is inserted into a bronchoscope 108 for access to a luminal network of the patient P. Specifically, catheter 102 of catheter guide assembly 106 may be inserted into a working channel of bronchoscope 108 for navigation through a patient's luminal network. A sensor 104 is located on the distal portion of the catheter 102. The position and orientation of sensor 104 relative to a reference coordinate system, and thus the distal portion of catheter 102, within an electromagnetic field can be derived. Catheter guide assemblies 106 are currently marketed and sold by Medtronic PLC under the brand names SUPERDIMENSION® Procedure Kits, or EDGE™ Procedure Kits, and are contemplated as useable with the disclosure.

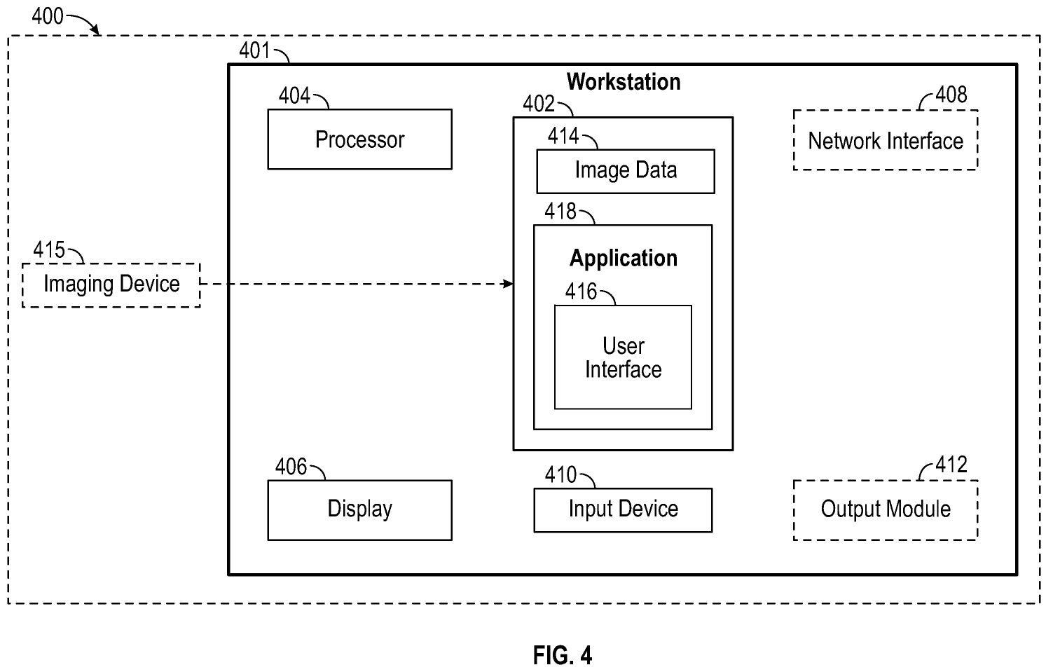

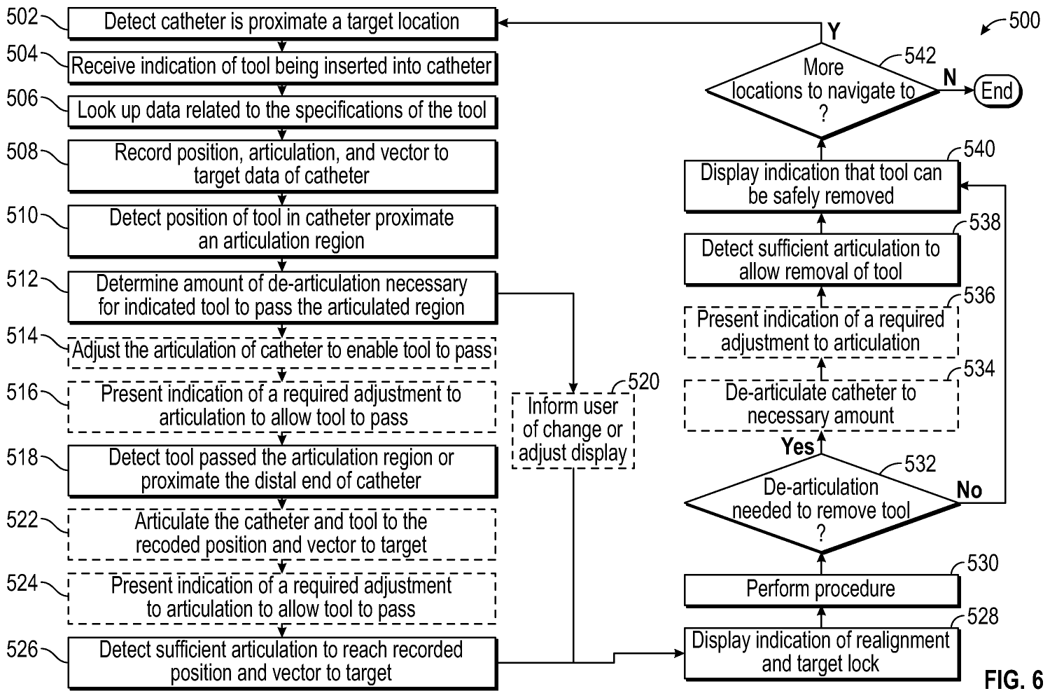

System 100 generally includes an operating table 112 configured to support a patient P, a bronchoscope 108 configured for insertion through patient P's mouth into patient P's airways; monitoring equipment 114 coupled to bronchoscope 108 (e.g., a video display, for displaying the video images received from the video imaging system of bronchoscope 108); a locating or tracking system 114 including a locating module 116, a plurality of reference sensors 18 and a transmitter mat 120 including a plurality of incorporated markers; and a computing device 122 including software and/or hardware used to facilitate identification of a target, pathway planning to the target, navigation of a medical device to the target, and/or confirmation and/or determination of placement of catheter 102, or a suitable device therethrough, relative to the target. Computing device 122 may be similar to workstation 401 of FIG. 4 and may be configured to execute the methods of the disclosure including the method of FIG. 6.

A fluoroscopic imaging device 124 capable of acquiring fluoroscopic or x-ray images or video of the patient P is also included in this particular aspect of system 100. The images, sequence of images, or video captured by fluoroscopic imaging device 124 may be stored within fluoroscopic imaging device 124 or transmitted to computing device 122 for storage, processing, and display. Additionally, fluoroscopic imaging device 124 may move relative to the patient P so that images may be acquired from different angles or perspectives relative to patient P to create a sequence of fluoroscopic images, such as a fluoroscopic video. The pose of fluoroscopic imaging device 124 relative to patient P and while capturing the images may be estimated via markers incorporated with the transmitter mat 120. The markers are positioned under patient P, between patient P and operating table 112 and between patient P and a radiation source or a sensing unit of fluoroscopic imaging device 124. The markers incorporated with the transmitter mat 120 may be two separate elements which may be coupled in a fixed manner or alternatively may be manufactured as a single unit. Fluoroscopic imaging device 124 may include a single imaging device or more than one imaging device.

Computing device 122 may be any suitable computing device including a processor and storage medium, wherein the processor is capable of executing instructions stored on the storage medium. Computing device 122 may further include a database configured to store patient data, CT data sets including CT images, fluoroscopic data sets including fluoroscopic images and video, fluoroscopic 3D reconstruction, navigation plans, and any other such data. Although not explicitly illustrated, computing device 122 may include inputs, or may otherwise be configured to receive, CT data sets, fluoroscopic images/video and other data described herein. Additionally, computing device 122 includes a display configured to display graphical user interfaces. Computing device 122 may be connected to one or more networks through which one or more databases may be accessed.

With respect to the planning phase, computing device 122 utilizes previously acquired CT image data for generating and viewing a three-dimensional model or rendering of patient P's airways, enables the identification of a target on the three-dimensional model (automatically, semi-automatically, or manually), and allows for determining a pathway through patient P's airways to tissue located at and around the target. More specifically, CT images acquired from previous CT scans are processed and assembled into a three-dimensional CT volume, which is then utilized to generate a three-dimensional model of patient P's airways. The three-dimensional model may be displayed on a display associated with computing device 122, or in any other suitable fashion. Using computing device 122, various views of the three-dimensional model or enhanced two-dimensional images generated from the three-dimensional model are presented. The enhanced two-dimensional images may possess some three-dimensional capabilities because they are generated from three-dimensional data. The three-dimensional model may be manipulated to facilitate identification of target on the three-dimensional model or two-dimensional images, and selection of a suitable pathway through patient P's airways to access tissue located at the target can be made. Once selected, the pathway plan, three-dimensional model, and images derived therefrom, can be saved and exported to a navigation system for use during the navigation phase(s).

With respect to the navigation phase, a six degrees-of-freedom electromagnetic locating or tracking system 114, or other suitable system for determining position and orientation of a distal portion of the catheter 102, is utilized for performing registration of the images and the pathway for navigation. Tracking system 114 includes the tracking module 116, a plurality of reference sensors 118, and the transmitter mat 120 (including the markers). Tracking system 114 is configured for use with a catheter 102 and particularly sensor 104.

Transmitter mat 120 is positioned beneath patient P. Transmitter mat 120 generates an electromagnetic field around at least a portion of the patient P within which the position of a plurality of reference sensors 118 and the sensor 104 can be determined with use of a tracking module 116. One or more of reference sensors 118 are attached to the chest of the patient P. Registration is generally performed to coordinate locations of the three-dimensional model and two-dimensional images from the planning phase, with the patient P's airways as observed through the bronchoscope 108, and allow for the navigation phase to be undertaken with knowledge of the location of the sensor 104.

Registration of the patient P's location on the transmitter mat 120 may be performed by moving sensor 104 through the airways of the patient P. More specifically, data pertaining to locations of sensor 104, while catheter 102 is moving through the airways, is recorded using transmitter mat 120, reference sensors 118, and tracking system 114. A shape resulting from this location data is compared to an interior geometry of passages of the three-dimensional model generated in the planning phase, and a location correlation between the shape and the three-dimensional model based on the comparison is determined, e.g., utilizing the software on computing device 122. In addition, the software identifies non-tissue space (e.g., air filled cavities) in the three-dimensional model. The software aligns, or registers, an image representing a location of sensor 104 with the three-dimensional model and/or two-dimensional images generated from the three-dimension model, which are based on the recorded location data and an assumption that sensor 104 remains located in non-tissue space in patient P's airways. Alternatively, a manual registration technique may be employed by navigating the bronchoscope 108 with the sensor 104 to pre-specified locations in the lungs of the patient P, and manually correlating the images from the bronchoscope to the model data of the three-dimensional model.

Still a further aspect of the disclosure relates to the catheter 102. The catheter 102 may include one or more pull-wires which can be used to manipulate the distal portion of the catheter. Pull-wire systems are known and used in a variety of settings including manual, power assisted, and robotic surgeries. In most catheter-based pull-wire systems at least one but up to six and even ten pull wires are incorporated into the catheter 102 and extend from proximate the distal end to a drive mechanism located at a proximal end. By tensioning at least one pull-wire the shape of the distal portion of the catheter can be manipulated. For example, in a two pull-wire system by relaxing one pull-wire and retracting an opposing pull-wire the catheter may be deflected in the direction of the retracting pull-wire. Though certain pull-wire systems are described here in detail, the disclosure is not so limited, and the manipulation of the catheter 102 may be achieved by a variety of means including concentric tube systems and others that enable movement of the distal end of the catheter 102. Further though a motor assisted/robotic system is described in detail, the same principals of extension and retraction of pull wires may be employed by manual manipulation means to change the shape of the distal portion of the catheter without departing from the scope of the disclosure.

Though described herein with respect to EMN systems using EM sensors, the instant disclosure is not so limited and may be used in conjunction with flexible sensor, ultrasonic sensors, or with other types of sensors. Additionally, the methods described herein may be used in conjunction with robotic systems such that robotic actuators drive the catheter 102 or bronchoscope 108 proximate the target as described in greater detail below.

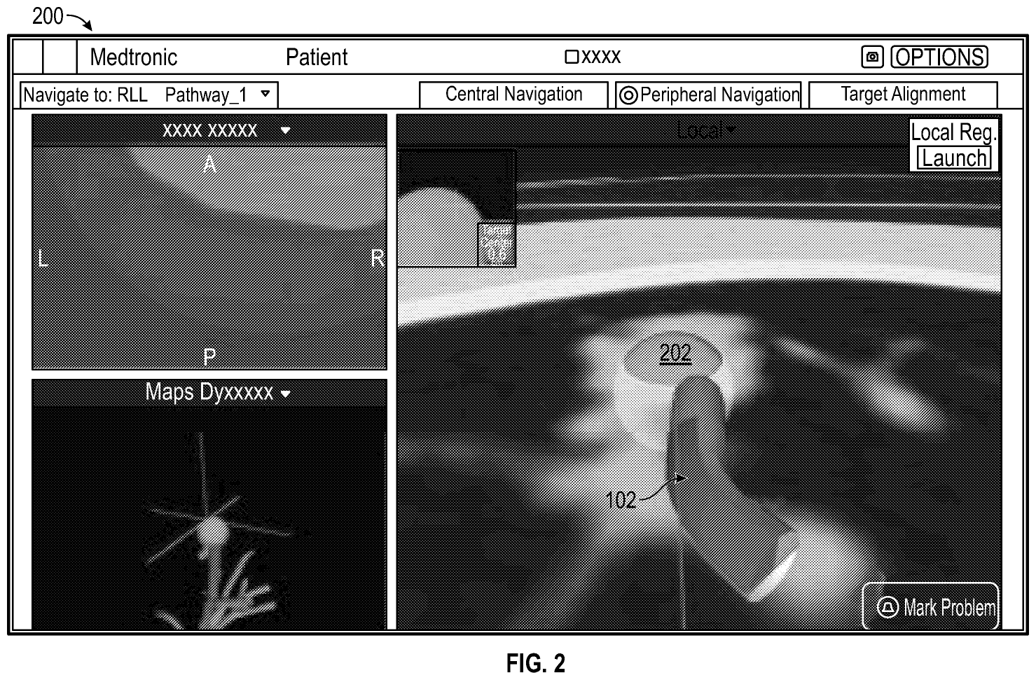

Following registration of the patient P to the image data and pathway plan, a user interface 200 as shown in FIG. 2A is displayed on the computing device 122 using the navigation software which sets forth the pathway that the clinician is to follow to reach the target. Once catheter 102 has been successfully navigated proximate, as shown in FIG. 2, the target 202 a local registration process may be performed for each target to reduce the CT-to-body divergence.

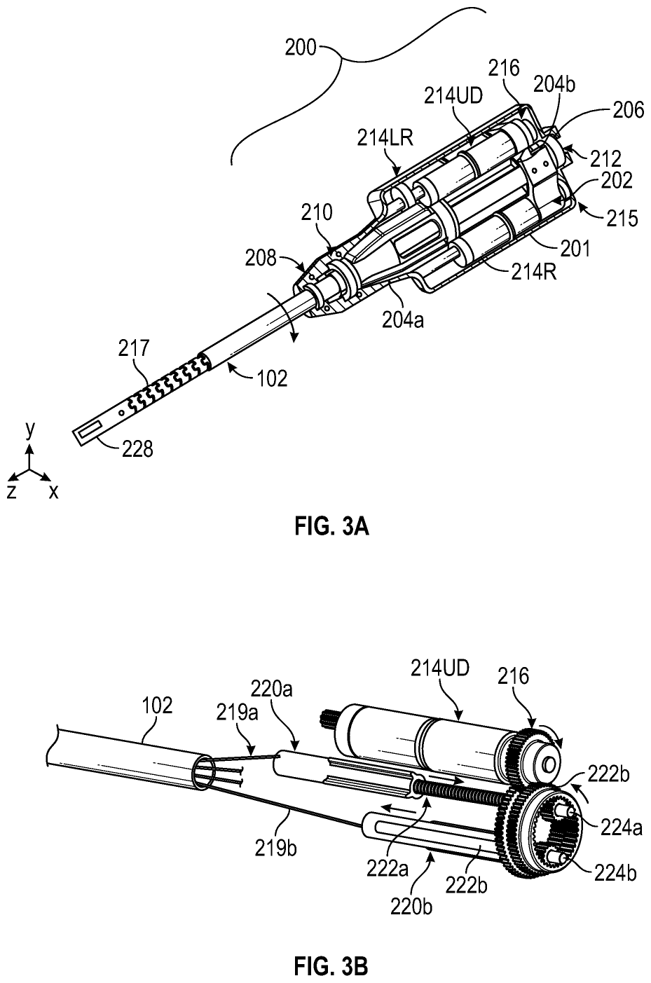

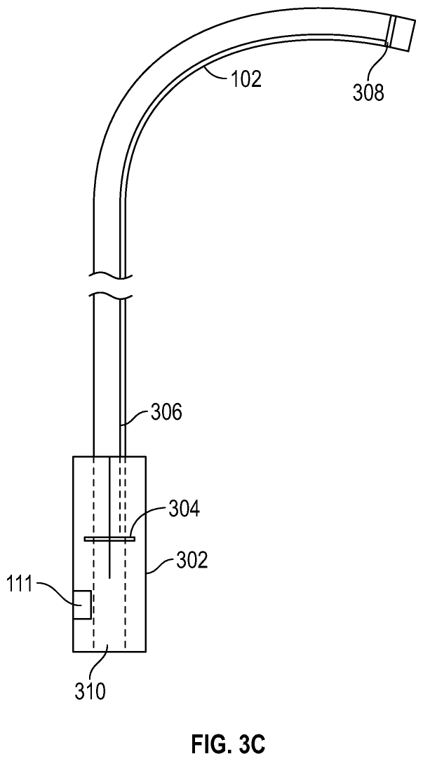

In procedures such as lung biopsy and treatment it is useful to ensure that the distal end of the catheter 102 is pointed directly at the target. In accordance with one aspect of the disclosure, a drive mechanism may receive signals derived by the computing device 122 to drive the catheter 102 (e.g., extend or retract pull-wires). One example of such a device can be seen in FIG. 3A which depicts a housing including three drive motors to manipulate a catheter extending therefrom in 5 degrees of freedom (e.g., left right, up, down, and rotation). Other types of drive mechanisms including fewer or more degrees of freedom and other manipulation techniques may be employed without departing from the scope of the disclosure.

As noted above, FIG. 3A depicts a drive mechanism 200 housed in a body 201 and mounted on a bracket 202 which integrally connects to the body 201. The catheter 102 connects to and in one embodiment forms an integrated unit with internal casings 204a and 204b and connects to a spur gear 206. This integrated unit is, in one embodiment rotatable in relation to the body 201, such that the catheter 102, internal casings 204 a-b, and spur gear 206 can rotate about shaft axis “z”. The catheter 102 and integrated internal casings 204 a-b are supported radially by bearings 208, 210, and 212. Though drive mechanism 200 is described in detail here, other drive mechanisms may be employed to enable a robot or a clinician to drive the catheter to a desired location without departing from the scope of the disclosure.

An electric motor 214R, may include an encoder for converting mechanical motion into electrical signals and providing feedback to the computing device 122. Further, the electric motor 214R (R indicates this motor if for inducing rotation of the catheter 102) may include an optional gear box for increasing or reducing the rotational speed of an attached spur gear 215 mounted on a shaft driven by the electric motor 214R. Electric motors 214LR (LR referring to left-right movement of an articulating portion 217 of the catheter 102) and 214UD (referring to up-down movement of the articulating portion 217), each motor optionally includes an encoder and a gearbox. Respective spur gears 216 and 218 drive up-down and left-right steering cables, as will be described in greater detail below. All three electric motors 214 R, LR, and UD are securely attached to the stationary frame 202, to prevent their rotation and enable the spur gears 215, 216, and 218 to be driven by the electric motors.

FIG. 3B depicts details of the mechanism causing articulating portion 217 of catheter 102 to articulate. Specifically, the following depicts the manner in which the up-down articulation is contemplated in one aspect of the disclosure. Such a system alone, coupled with the electric motor 214UD for driving the spur gear 216 would accomplish articulation as described above in a two-wire system. However, where a four-wire system is contemplated, a second system identical to that described immediately hereafter, can be employed to drive the left-right cables. Accordingly, for ease of understanding just one of the systems is described herein, with the understanding that one of skill in the art would readily understand how to employ a second such system in a four-wire system. Those of skill in the art will recognize that other mechanisms can be employed to enable the articulation of a distal portion of a catheter and other articulating catheters may be employed without departing from the scope of the disclosure.

To accomplish up-down articulation of the articulating portion 217 of the catheter 102, steering cables 219a-b may be employed. The distal ends of the steering cables 219a-b are attached to, or at, or near the distal end of the catheter 102. The proximal ends of the steering cables 219a-b are attached to the distal tips of the posts 220a, and 220b. The posts 220a and 220b reciprocate longitudinally, and in opposing directions. Movement of the posts 220a causes one steering cable 219a to lengthen and at the same time, opposing longitudinal movement of post 220b causes cable 219b to effectively shorten. The combined effect of the change in effective length of the steering cables 219a-b is to cause joints a forming the articulating portion 217 of catheter 102 shaft to be compressed on the side in which the cable 219b is shortened, and to elongate on the side in which steering cable 219 a is lengthened.

The opposing posts 220a and 1220b have internal left-handed and right-handed threads, respectively, at least at their proximal ends. Housed within casing 1204b are two threaded shafts 222a and 222b, one is left-hand threaded and one right-hand threaded, to correspond and mate with posts 220a and 220b. The shafts 222a and 222b have distal ends which thread into the interior of posts 220a and 220a and proximal ends with spur gears 224a and 224b. The shafts 222a and 222b have freedom to rotate about their axes. The spur gears 224a and 224b engage the internal teeth of planetary gear 226. The planetary gear 226 also includes external teeth which engage the teeth of spur gear 218 on the proximal end of electric motor 214UD.

To articulate the catheter in the upwards direction, a clinician may activate via an activation switch (not shown) for the electric motor 214UD causing it to rotate the spur gear 218, which in turn drives the planetary gear 226. The planetary gear 226 is connected through the internal gears 224a and 224b to the shafts 222a and 222b. The planetary gear 226 will cause the gears 224a and 224b to rotate in the same direction. The shafts 222a and 222b are threaded, and their rotation is transferred by mating threads formed on the inside of posts 220a and 220b into linear motion of the posts 220a and 220b. However, because the internal threads of post 220a are opposite that of post 220b, one post will travel distally and one will travel proximally (i.e., in opposite directions) upon rotation of the planetary gear 226. Thus, the upper cable 219a is pulled proximally to lift the catheter 102, while the lower cable 219b must be relaxed. As stated above, this same system can be used to control left-right movement of the end effector, using the electric motor 214LR, its spur gear 216, a second planetary gear (not shown), and a second set of threaded shafts 222 and posts 220 and two more steering cables 219. Moreover, by acting in unison, a system employing four steering cables can approximate the movements of the human wrist by having the three electric motors 214 and their associated gearing and steering cables 219 computer controlled by the computing device 122.

Though generally described above with respect to receiving manual inputs from a clinician as might be the case where the drive mechanism is part of a hand-held catheter system, the disclosure is not so limited. In a further embodiment, the drive mechanism 200 is part of a robotic system for navigating the catheter 102 to a desired location within the body. In accordance with this disclosure, in instances where the drive mechanism is part of a robotic catheter drive system, the position of the distal portion of the catheter 102 may be robotically controlled. In such an instance the computing device 122, which determines the position of the target and the catheter 102

The drive mechanism may receive inputs from computing device 122 or another mechanism through which the surgeon specifies the desired action of the catheter 102. Where the clinician controls the movement of the catheter 102, this control may be enabled by a directional button, a joystick such as a thumb operated joystick, a toggle, a pressure sensor, a switch, a trackball, a dial, an optical sensor, and any combination thereof. The computing device responds to the user commands by sending control signals to the motors 214. The encoders of the motors 214 provide feedback to the control unit 24 about the current status of the motors 214.

While FIGS. 3A and 3B depict a complex articulation mechanism, this disclosure is not so limited. FIG. 3C depicts a catheter 102 having a handle 302 on a proximal end. The handle 302 includes a slider 304 that may be manually manipulated distally and proximally. Operatively connected to the slider 304 is a pull-wire 306. The pull-wire 306 may extend through a lumen (not shown) formed in the wall of the catheter 102. The pull-wire 306 terminates at a pull ring 308. The pull ring 308 may be integrally formed with the catheter 102 such that retraction proximally of the slider 304 causes the pull-wire 306 to retract and therewith cause the catheter 102 to articulate. Advancement of the slider 304 allows the pull-wire 306 to relax and the catheter 102 to return to a less articulate state. The elastic properties of the materials of the catheter 106 force the return in shape once the pressure applied by the pull-wire 306 is reduced or eliminated. A lumen 310 which extends through the handle 302 and the catheter 102.

Reference is now made to FIG. 4, which is a schematic diagram of a system 400 configured for use with the methods of the disclosure including the method of FIG. 4. System 400 may include a workstation 401, and optionally a fluoroscopic imaging device or fluoroscope 415. In some embodiments, workstation 401 may be coupled with fluoroscope 415, directly or indirectly, e.g., by wireless communication. Workstation 401 may include a memory 402, a processor 404, a display 406 and an input device 410. Processor or hardware processor 404 may include one or more hardware processors. Workstation 401 may optionally include an output module 412 and a network interface 408. Memory 402 may store an application 418 and image data 414. Application 418 may include instructions executable by processor 404 for executing the methods of the disclosure including the method of FIG. 4.

Application 418 may further include a user interface 416. Image data 414 may include the CT scans, the generated fluoroscopic 3D reconstructions of the target area and/or any other fluoroscopic image data and/or the generated one or more slices of the 3D reconstruction. Processor 404 may be coupled with memory 402, display 406, input device 410, output module 412, network interface 408 and fluoroscope 415. Workstation 401 may be a stationary computing device, such as a personal computer, or a portable computing device such as a tablet computer. Workstation 401 may embed a plurality of computer devices.

Memory 402 may include any non-transitory computer-readable storage media for storing data and/or software including instructions that are executable by processor 404 and which control the operation of workstation 401 and, in some embodiments, may also control the operation of fluoroscope 415. Fluoroscope 415 may be used to capture a sequence of fluoroscopic images based on which the fluoroscopic 3D reconstruction is generated and to capture a live 2D fluoroscopic view according to this disclosure. In an embodiment, memory 402 may include one or more storage devices such as solid-state storage devices, e.g., flash memory chips. Alternatively, or in addition to the one or more solid-state storage devices, memory 402 may include one or more mass storage devices connected to the processor 404 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 404. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 1001.

Application 418 may, when executed by processor 404, cause display 406 to present user interface 416. User interface 416 may be configured to present to the user a single screen including a three-dimensional (3D) view of a 3D model of a target from the perspective of a tip of a medical device, a live two-dimensional (2D) fluoroscopic view showing the medical device, and a target mark, which corresponds to the 3D model of the target, overlaid on the live 2D fluoroscopic view, as shown, for example, in FIG. 2. User interface 416 may be further configured to display the target mark in different colors depending on whether the medical device tip is aligned with the target in three dimensions.

Network interface 408 may be configured to connect to a network such as a local area network (LAN) consisting of a wired network and/or a wireless network, a wide area network (WAN), a wireless mobile network, a Bluetooth network, and/or the Internet. Network interface 408 may be used to connect between workstation 401 and fluoroscope 415. Network interface 408 may be also used to receive image data 414. Input device 410 may be any device by which a user may interact with workstation 401, such as, for example, a mouse, keyboard, foot pedal, touch screen, and/or voice interface. Output module 412 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

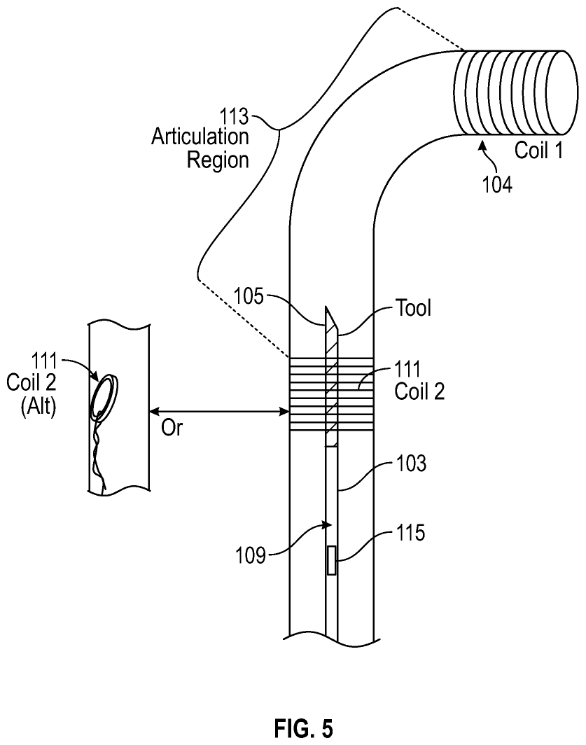

Further features of the catheter 102 can be seen with respect to FIG. 5. As with other catheters 102 described herein, the catheter 102 of FIG. 5 includes a sensor 104 at the distal end for determination of the location of the distal end of the catheter 102. In addition to the sensor 104, a second sensor 111 maybe employed. The sensor 111, which may be a coil wrapped around the lumen of the catheter 102 or a coil placed on the wall of the lumen of the catheter 102, when placed in the electromagnetic field generated by the transmitter mat 120 generates a current. As a ferrous or other magnetically distorting material passes proximate the sensor 111, the electromagnetic field proximate the sensor 111 is distorted. In accordance with one aspect of the disclosure, as described in greater detail below, this distortion can be sensed and used as a signal to indicate that the metal tip 105 of the tool 103 is proximate an articulating region 113.

As an alternative to the second sensor 111 being located along the catheter 102, the second sensor 111 may be an optical sensor as depicted in FIG. 3C mounted on the proximal end of the catheter 102, for example as part of the handle 302. As a tool 103 is advanced through the lumen 310 markings on the tool can be read or sensed and a determination of how far along the catheter 102, the tool 105 has traversed. In accordance with some aspects of the disclosure the pattern on the tool 103 can be sensed by the optical sensor 111 to determine the type of tool 103 being inserted into the catheter 102.

Another aspect of the disclosure is related to a chip 115 that is associated with the tool 103. The chip 115 which may be an RFID chip is read by a reader (not shown) associated with the computing device 122 and an application 418 that reads the chip 115 and determines the type of tool 105 that is being employed. As will be described in greater detail below the application 418, upon determining the type of the tool 103 can refer to a look-up table or other data source detailing the specifications of the tool 103 including amount of articulation of the catheter 102 the tool 103 can traverse without impacting the sidewalls of the catheter 102, the amount of articulation that will still allow the insertion of the tool 103 without resulting in friction preventing advancement of the tool. The look-up table may also have information regarding the flexibility properties of the tool 103. Other items of data may also be stored therein. As an alternative to an RFID chip, the look-up table or other data store may be accessed by a user via the user interface 416 and for example drop-down menus which are presented during certain portions of a procedure.

Regardless of the drive mechanism 200 or articulation system 300 employed to articulate the catheter 102, a further aspect of the disclosure is directed to a method 500. The method begins at step 502 where position of the sensor 104 on the catheter 102 is determined by the navigation application 418 on computing device 122 to be proximate a target location, as shown in FIG. 2. As will be appreciated the placement of the distal end of the catheter 102 proximate the target location often requires articulation, sometimes significant articulation of the catheter 102 to orient the opening in the distal end of the catheter 102 such that a tool 103 that is extended from the catheter 102 can interact with the target tissue (e.g., a lesion or tumor, or tissue suspected of being a lesion or tumor).

At some point during the navigation of the catheter 102 through a tortuous path, for example of the airways a patient, to reach a target location a tool 103 is inserted into the catheter 102. The type of tool 103 being inserted is identified at step 504. This identification may be achieved by selecting it from a drop-down menu on a user interface, may be read by an optical sensor 111 associated with the catheter 102, or a sensed from chip 115 via a chip reader associated with the computing device 122. Regardless of which system is employed to identify the tool 103, the identification of the tool 103 results in data regarding the tool 103 to be accessed at step 506. This may be accessing a look-up table or other data store associated with the computing device 122 and useable by the application 418 to determine aspects such as the maximum articulation of a catheter 102 the tool 103 can traverse, an amount the articulation needs to be de-articulated to allow the tool 103 to pass the articulation region 113, an amount the added stiffness of the tool 103 will change the curvature of the articulation of the catheter 102, an amount of additional force needed to achieve an articulation of the tool 103 similar that that of the catheter 102, the amount of articulation that the catheter 102 can experience without creating a friction fit that prevents the advancement of the tool 103 even when past the articulation region 113, as well as other data related to the physical properties of the tool and its interaction with the catheter 102.

At step 508 the position, amount of articulation, and a vector to a target are recorded by the application 418. This recordation provides a point and an orientation to which the distal end of the catheter should be articulated to in order to allow the tool 103 to interact with the target location (e.g., tumor or lesion). As the tool 103 is advanced through the catheter 102 its position within the catheter 102 can be detected either through the use of a sensor 111 to detect disruptions in the electromagnetic field or through the use of the optical sensor 111 in the handle 302. At some point of advancement, the tool 103 is detected proximate the articulation region at step 510. For example, this may be achieved by the use of multiple markings on the tool 103 at known intervals and determination of the distance the tool 103 has traveled within the catheter 102 by counting the number of such markings to pass the optical sensor 111 (e.g., an odometry measurement). Alternatively, an optical sensor 111 pair may be employed to detect the distance that the tool 103 travels through the catheter. The optical sensor 111 pair (one emitter and one detector) transmits light onto the tool 103. Based on a comparison of the detected properties of successive images captured by the detector, a determination can be made of the speed and distance that the tool 105 has travelled within the catheter 102. Where the optical sensor 111 pair is a high-performance optical sensor there is no need for inclusion of any special patterns or other features on the exterior surface of the tool 105. In some instances, accurate distance and speed measurements can be made based on the minute variations in the surface of the t001 105, much the same way that an optical mouse can be used with a computer.

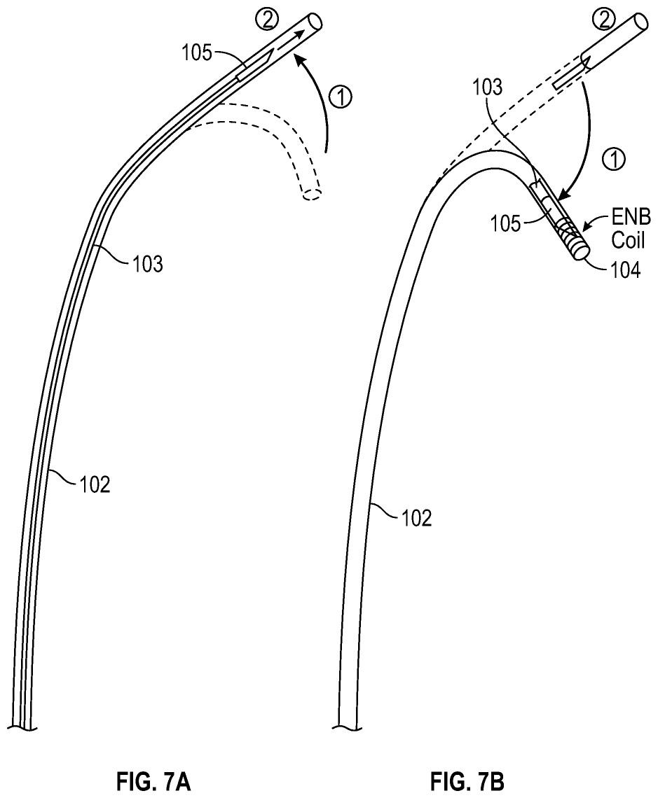

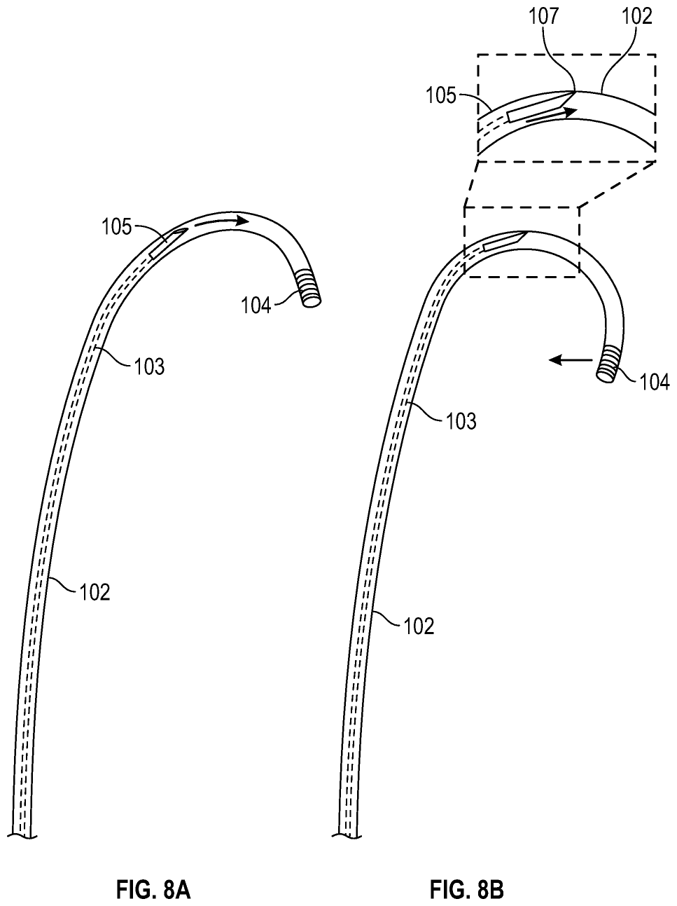

Once the tool 103 is detected proximate the articulation zone 113 at step 510, at step 512 the application 418 can determine an amount of de-articulation of the catheter 102 to allow the tool 103 to pass the articulation portion 113. Following the determination at step 512, if a robotic or motorized catheter 102 as depicted in FIGS. 3A and 3B is being utilized the application 418 may at step 514 automatically drive the catheter 102 to relax the force applied to the pull wire 306 or steering cables 219 to reduce the amount of articulation of the catheter 102 to allow the tool to pass the articulation region 113 and arrive at the distal end of the catheter 102 without damaging an internal surface of the catheter 102. An example of this can be seen in FIG. 7A, showing the catheter 102 moving from articulated position 1 to de-articulated position 2 so that tool 103 can be advanced to the distal end of the catheter 102. As an alternative the user interface 416 of the application 418 may display an amount of de-articulation at step 516 to enable passage of the tool 103. Whether manually operated via a slider 304, as shown in FIG. 3C, or motor operated, as depicted in FIGS. 3A and 3B, the change in articulation required can be displayed on the user interface 416 awaiting the user to undertake the necessary steps to reduce the force applied to the pull wire 306 or the steering cables 219 to achieve the desired de-articulation. Once de-articulation is achieved, the tool 103 can be advanced passed the articulation region 113, and the proximity of the tool 103 to the distal end of the catheter 102 can be detected at step 518. While the de-articulation and advancement of the tool 103 are undertaken, the images displayed on the user interface 416 can be adjusted at step 520. Such a step of obscuring the images may be particularly beneficial where a robotic or automatic motor operated systems is being employed. This adjustment to the user interface 416 may be to obscure the movement of the catheter 102 during the de-articulation process. Obscuring the movement of the catheter 102 may prevent the user from making the false assumption that the de-articulation is something that needs to be overcome or is an undesirable drift of the catheter 102. For example, the user interface may be frozen during this process or obscured by an indicator stating that the de-articulation process is being undertaken to allow the tool 103 to pass through the articulation region 113. Alternatively, the update rate of the images depicted on the user interface may simply be slowed and an indicator displayed on the user interface 416 to alert the user that a de-articulation process is being undertaken to allow the tool 103 to pass through the articulation region 113. However, step 520 is optional and all of the movement of the catheter 102 may be accurately displayed in the user interface 416 throughout the process to accurately display the movement of the catheter 102 on the user interface 416 so that the user may follow the de-articulation and re-articulation process (e.g., steps 512-528).

After determination that the tool 103, and particularly the metal tip 105 has passed the articulation region 113, at step 522 the catheter is re-articulated back to the same location and orientation with the same vector to the target location as determined at step 508 prior to passage of the tool 103 through the articulation region 113. This may be particularly employed where robotic or motorized articulation mechanisms, for example as described with respect to FIGS. 3A and 3B. Alternatively, where a manually operated systems is employed whether using the slider 304 on a handle 302 as shown in FIG. 3C or a motorized systems shown in FIGS. 3A and 3B but receiving manual inputs, an indication is provided on the user interface 416 at step 524 directing the user to adjust the articulation of the catheter 102 and the tool 103 to re-orient the distal end of the catheter 102 and the tool 103 such that the opening at the distal end of the catheter 102 is re-articulated back to the same location and orientation with the same vector to the target location as determined at step 508 prior to passage of the tool 103 through the articulation region 113. An example of the re-articulation process can be seen in FIG. 7A from position 2 to position 1, but with the tool 103 located proximate the distal end of the catheter 102.

At step 526 the position and orientation of the distal end of the catheter 102 and tool 103 at the location and orientation with the same vector to the target location as determined at step 508 is detected. At step 528 an indication of realignment and a lock on the target location can be displayed on the user interface 416. Once achieved, the user is free to perform a procedure at the target location, e.g., biopsy or therapy or some other procedure relevant to the patient at step 530. After performance of the procedure, at step 532 a determination can be made whether de-articulation 532 is required for removal of the tool 105. If not the process advances to step 540.

If, however, articulation is needed at step 532, for example based on the data referenced in step 506, the catheter 102 may be de-articulated (e.g., using the automated devices of FIGS. 3A and 3B) at step 534. Alternatively, an indication of the amount of de-articulation necessary can be displayed on the user interface 416 at step 536 allowing the user to either drive the motorized systems of FIGS. 3A and 3B or manipulate the slider 304. At step 538 sufficient de-articulation of the catheter 102 is detected and at step 540 an indication can be displayed on the user interface 416 that the tool can safely be removed. At step 542 a determination is made as to whether there are additional locations within the body to navigate to. If no other locations require navigation to the procedure may end. Alternatively, the catheter 102 needs to be navigated to other target locations, the catheter 102 can be so navigated and detection of the catheter 102 proximate the target can be again detected, and the process repeated until all target locations have been navigated to.

While detailed embodiments are disclosed herein, the disclosed embodiments are merely examples of the disclosure, which may be embodied in various forms and aspects. For example, embodiments of an electromagnetic navigation system, which incorporates the target overlay systems and methods, are disclosed herein; however, the target overlay systems and methods may be applied to other navigation or tracking systems or methods known to those skilled in the art. Therefore, specific structural and functional details disclosed herein are not to be interpreted as limiting, but merely as a basis for the claims and as a representative basis for teaching one skilled in the art to variously employ the disclosure in virtually any appropriately detailed structure.

We claim:

1. A system for navigation of a catheter to a location within a patient comprising:
a catheter including a lumen and a position sensor at or adjacent a distal portion, a proximity sensor, and an articulating region;
an articulation mechanism including at least one pull-wire for articulating the articulating region of the catheter;
a tool for performing a procedure at a target site and translatable within the lumen of the catheter, wherein the proximity sensor is configured to detect the tool is proximate the articulating region; and
an application stored in a memory and executed by a processor of a computing device, wherein the application when executed performs steps of:
determine properties of the tool based on a received an indication of a type of tool;
record a position, articulation and vector to a target of the catheter;
detect the tool in proximity to the articulation region;
determine an amount of de-articulation necessary for the tool to pass through the articulation region;
adjust the articulation of the catheter;
detect the tool has passed the articulation region and is proximate a distal portion of the catheter;
articulate the catheter to the recorded position;
detect that the catheter has returned to the recorded position; and
display an indication of realignment on a user interface.

2. The system of claim 1, wherein the application further performs a step of presenting an indication on the user interface of a required amount of de-articulation necessary to allow the tool to pass through the articulation region.

3. The system of claim 1, wherein the articulation mechanism is a motorized mechanism.

4. The system of claim 3, wherein the motorized mechanism is robotically controlled.

5. The system of claim 3, wherein the motorized mechanism is manually operated.

6. The system of claim 1, wherein the articulation mechanism is manually operated.

7. The system of claim 1, wherein the application further performs a step of determining if de-articulation is required to remove the tool.

8. The system of claim 7, wherein the application further perform steps of de-articulating the catheter and displaying an indication on the user interface that the tool can be safely removed.

9. A method of placement of a tool proximate a target comprising:
receiving an image data set of a patient;
generating a 3D model of a luminal network from the image data set;
generating a pathway to a target location within the 3D model of the luminal network;
detecting a catheter including a position sensor has been navigated to a position within the luminal network proximate the target location;
receiving an indication of a type of tool being inserted into the catheter;
determining specification data related to the type of tool being inserted into the catheter;
recording the position, articulation, and a vector to the target location from a distal end of the catheter to the target location;
detecting the position of the tool proximate an articulation region of the catheter;
determining an amount of de-articulation necessary for the catheter to pass through the articulation region;
adjusting the articulation of the catheter;
detecting passage of the tool through the articulation region;
articulating the catheter to the recorded position, articulation and vector to the target; and
displaying on a user interface an indication of realignment of the catheter with the target location.

10. The method of claim 9, wherein determining specification data includes querying a look-up table in which the specification data is recorded.

11. The method of claim 9, further comprising presenting an indication on the user interface of an amount of de-articulation necessary to allow the tool to pass through the catheter.

12. The method of claim 11, further comprising manually manipulating a motor driven pull-wire system to de-articulate the catheter to achieve the presented amount of de-articulation.

13. The method of claim 11, further comprising manually adjusting a pull-wire to achieve the presented amount of de-articulation.

14. The method of claim 9, further comprising robotically driving a motor driven pull-wire system to de-articulate the catheter.

15. The method of claim 9 further comprising determining if de-articulation is required to remove the tool.

16. The method of claim 15 further comprising de-articulating the catheter and displaying an indication on the user interface that the tool can be safely removed.

17. The method of claim 16, wherein the amount of de-articulation necessary is determined based on the determined specification data related to the type of tool inserted into the catheter.

18. A catheter system comprising:
a catheter having a lumen, a position sensor located at or adjacent a distal portion, a proximity sensor, and at least one pull-wire configured to articulate an articulation region;
a tool including a metal tip at a distal end;
a proximity sensor associated with the catheter and configured to detect a presence of the tool proximate the articulation region;
an articulation mechanism configured to apply pressure to the pull-wire to articulate and de-articulate the catheter; and
a computing device, storing in a memory an application executable by a processor, the application when executed determining steps of:
determining that the tool cannot pass through the articulation region based on the articulation of the catheter and properties of the tool;
de-articulating the catheter to allow the tool to pass through the articulation region; and
re-articulating the catheter to a position and orientation of the catheter prior to de-articulation.

19. The catheter system of claim 18, wherein the proximity sensor is an optical sensor configured to read markings on the tool.

20. The catheter system of claim 18, wherein the proximity sensor is an electromagnetic field sensor configured to detect interference in the electromagnetic field as the metal tip is advanced into proximity of the proximity sensor.
